SEQUENCE LISTING (10 sequences)

SEQ ID NO: 1 - DNA, 1578 bp, Escherichia coli
  source 1..1578: mol_type = other DNA
atggcacatatgaccaatgtgcgccgggtgtttaatgaattgattggcgacgatgaaagtgaaactcaggaagagtcgctgtcggaacagtggcgtgagctgtggcaggatgcgttgcaggaagatgacactacgccagtgctggcgcatcttagcgaggatgatcgcaaacaggtgctaacgctgattgccgatttccgcaaagagctggataagcgcaccatcgggccgcgaggacgtcaggtgctcgaccatctgatgccgcatctgctaagtgatgtctgtgcgcgtgaagacgctgccgttacgctgtcgcgcattaccgccttgctggtggggattgttacccgcaccacctatttagaattgctcagtgaattccccgcggcgcttaaacatttgatttctctgtgtgccgcgtcgccgatgattgccagccagctggcgcgttatccattattgctggatgaattgctcgatccaaacaccctttaccagccgacggcgaccgatgcctaccgcgatgagttgcgccagtatttgctgcgcgtgccggaagatgacgaagagcaacagcttgaggcgctgcgtcagttcaaacaggcgcagctgttacgcatcgccgcagcggatatcgccggtacgctaccggtgatgaaagtgagcgatcacttaacctggctggcggaagccatgatagatgccgtcgttcagcaggcgtgggttcaaatggttgcccgctacggtaagccgaatcacctgaacgaacgcgaagggcgtggttttgcggtggtcggctacggcaagctgggcggctgggagttaggctacagttccgatcttgaccttatcttcctccatgattgcccaatggatgcgatgactgacggtgagcgggaaatcgacgggcggcagttttatctgcgtctggcgcaacgcattatgcatctgttcagtacgcgtacctcttccggcattttgtatgaagtggatgctcgactgcgtccgtccggggcggcgggaatgctggtgacatccgcagaagcatttgccgattatcagaaaaacgaggcctggacgtgggaacatcaggcgctggtgcgtgcgcgtgtagtgtacggcgatccgcagctcaccgcgcactttgacgcagtgcgtcgcgagattatgacgctgccgcgtgaaggtaaaactctgcaaacggaagtgcgggaaatgcgcgagaaaatgcgcgctcatctcggcaataaacatcgcgatcgctttgatatcaaagctgatgaagggggaattaccgatatcgaatttattacccaatatctggtgttgcgctacgctcatgaaaaaccgaagttaacgcgctggtcagacaacgtgcgtattctggaactactggcgcaaaacgacattatggaagagcaggaagcgatggcgctgacccgtgcttacactacgcttcgcgatgaacttcatcatctggcattacaggaattgccgggccatgtgtcggaggattgcttcaccgcagagcgtgaactggtgcgggcaagctggcagaagtggctggtggaagaatga

SEQ ID NO: 2 - DNA, 1020 bp, Escherichia coli
  source 1..1020: mol_type = other DNA
atggacgaagagcaacagcttgaggcgctgcgtcagttcaaacaggcgcagctgttacgcatcgccgcagcggatatcgccggtacgctaccggtgatgaaagtgagcgatcacttaacctggctggcggaagccatgatagatgccgtcgttcagcaggcgtgggttcaaatggttgcccgctacggtaagccgaatcacctgaacgaacgcgaagggcgtggttttgcggtggtcggctacggcaagctgggcggctgggagttaggctacagttccgatcttgaccttatcttcctccatgattgcccaatggatgcgatgactgacggtgagcgggaaatcgacgggcggcagttttatctgcgtctggcgcaacgcattatgcatctgttcagtacgcgtacctcttccggcattttgtatgaagtggatgctcgactgcgtccgtccggggcggcgggaatgctggtgacatccgcagaagcatttgccgattatcagaaaaacgaggcctggacgtgggaacatcaggcgctggtgcgtgcgcgtgtagtgtacggcgatccgcagctcaccgcgcactttgacgcagtgcgtcgcgagattatgacgctgccgcgtgaaggtaaaactctgcaaacggaagtgcgggaaatgcgcgagaaaatgcgcgctcatctcggcaataaacatcgcgatcgctttgatatcaaagctgatgaagggggaattaccgatatcgaatttattacccaatatctggtgttgcgctacgctcatgaaaaaccgaagttaacgcgctggtcagacaacgtgcgtattctggaactactggcgcaaaacgacattatggaagagcaggaagcgatggcgctgacccgtgcttacactacgcttcgcgatgaacttcatcatctggcattacaggaattgccgggccatgtgtcggaggattgcttcaccgcagagcgtgaactggtgcgggcaagctggcagaagtggctggtggaagaatga

SEQ ID NO: 3 - DNA, 1671 bp, Azospirillum brasilense
  source 1..1671: mol_type = other DNA
atgctcggccgggtggaggaccgctacgccgagctgttcgaggaggcgccgtcgctgtccggccccggcaacctcgtcttcaccggcaccgacgacgaccccggcacggtgaagacgctggccggcatgggctaccgcgaccccagccgggtcatcgccgtggtctccacctggcaccgcgggcgctaccgctccacccgctcgggccgcgcgcgggagctgctgaccgagctggtgccggccatgctgaacgagctggcgaagacccccgccccggacgacgcgctggttaagttcgacagcttcctggagcggcttccggcgggggtcgggctgttctcgctgttcatcgccaacccctggctgctcgccctggtcgcggagatcatgggcacggcgccgcagctggccgagacgttgtcgcgcaacccgtcgctgctcgacgccgtgctgtcgcccgacttcttcgacccgctgcccgacgcggcggggctgacgccggagtaccagcgcttcatcgccggggcgcacaacttcgaggatgtgctgaccctgtcgcggcgctggaccaacgaccagcgcttccgcgccggggcgcacatcctgcgcggcatcaccgacggcgaccgctgcggccccttcctcgccgatctggccgacgtggtggtgccggagctggccgcccgcgtggaggaggagttcgccgcccgccacggccgcatccccggcggcgcctgggtggtggtggcgatgggcaagctaggcagccggcagctcaccatcacctccgacatcgacctgatcgtggtctacgaggtgccgccgggcacccgccagtcggacggagccaagccgctggcccccaacgagtattacatcaagctgacgcagcgcctgaccaacgccatcaccgccccgatggccgacgggcggctgtacgaggtggacatgcggctgcgcccgtcgggcaacgccggtccgctcgccaccgcgctggacgccttcaccgcctatcaggccaaggatgcctggacgtgggagcacatggccctgacccgcgcccgcgtcatcggcagtgattccggcggcggcgatccggcgctgggtcataaggtcgagtcggcgatccgcggtgtgctgaccggcccgcgcgacccggccaaggtgctgcgggacgtcgccgacatgcgccgccgcatcgacaaggagttcggcaccaccaacccgtggaacgtcaaatacgcccgcggcggcctgatcgacatcgagttcacggcccagtatctccagctccgtcacggccacgcgcatccggacatcctgtccatcgccaccagccgcgccctgctcaacgccgctgcggccgggctgctggcgccggaggtggcggaggagctggtggcgacgctgaagctgtggcggcgggtgcagggcttcctgcgcctgaccaccgacggcgtgctcgatccgcggcaggtttcgcccaccctgcgggagggcctctcgcgcgccgccttcccggacgaggagccggcggttgacttcgccgcgctcgacagcagaatccgggacatcgccgcccgcgcccaccgccatttcgtggcgctggtcgaggagccggcgtcaaggctgcctcccccagagaccaacgaagaagccaaactcccatga

SEQ ID NO: 4 - DNA, 1143 bp, Azospirillum brasilense
  source 1..1143: mol_type = other DNA
atgaacttcgaggatgtgctgaccctgtcgcggcgctggaccaacgaccagcgcttccgcgccggggcgcacatcctgcgcggcatcaccgacggcgaccgctgcggccccttcctcgccgatctggccgacgtggtggtgccggagctggccgcccgcgtggaggaggagttcgccgcccgccacggccgcatccccggcggcgcctgggtggtggtggcgatgggcaagctaggcagccggcagctcaccatcacctccgacatcgacctgatcgtggtctacgaggtgccgccgggcacccgccagtcggacggagccaagccgctggcccccaacgagtattacatcaagctgacgcagcgcctgaccaacgccatcaccgccccgatggccgacgggcggctgtacgaggtggacatgcggctgcgcccgtcgggcaacgccggtccgctcgccaccgcgctggacgccttcaccgcctatcaggccaaggatgcctggacgtgggagcacatggccctgacccgcgcccgcgtcatcggcagtgattccggcggcggcgatccggcgctgggtcataaggtcgagtcggcgatccgcggtgtgctgaccggcccgcgcgacccggccaaggtgctgcgggacgtcgccgacatgcgccgccgcatcgacaaggagttcggcaccaccaacccgtggaacgtcaaatacgcccgcggcggcctgatcgacatcgagttcacggcccagtatctccagctccgtcacggccacgcgcatccggacatcctgtccatcgccaccagccgcgccctgctcaacgccgctgcggccgggctgctggcgccggaggtggcggaggagctggtggcgacgctgaagctgtggcggcgggtgcagggcttcctgcgcctgaccaccgacggcgtgctcgatccgcggcaggtttcgcccaccctgcgggagggcctctcgcgcgccgccttcccggacgaggagccggcggttgacttcgccgcgctcgacagcagaatccgggacatcgccgcccgcgcccaccgccatttcgtggcgctggtcgaggagccggcgtcaaggctgcctcccccagagaccaacgaagaagccaaactcccatga

SEQ ID NO: 5 - DNA, 1575 bp, Azospirillum brasilense
  source 1..1575: mol_type = other DNA
atgcccggcacggtgaagacgctggccggcatgggctaccgcgaccccagccgggtcatcgccgtggtctccacctggcaccgcgggcgctaccgctccacccgctcgggccgcgcgcgggagctgctgaccgagctggtgccggccatgctgaacgagctggcgaagacccccgccccggacgacgcgctggttaagttcgacagcttcctggagcggcttccggcgggggtcgggctgttctcgctgttcatcgccaacccctggctgctcgccctggtcgcggagatcatgggcacggcgccgcagctggccgagacgttgtcgcgcaacccgtcgctgctcgacgccgtgctgtcgcccgacttcttcgacccgctgcccgacgcggcggggctgacgccggagtaccagcgcttcatcgccggggcgcacaacttcgaggatgtgctgaccctgtcgcggcgctggaccaacgaccagcgcttccgcgccggggcgcacatcctgcgcggcatcaccgacggcgaccgctgcggccccttcctcgccgatctggccgacgtggtggtgccggagctggccgcccgcgtggaggaggagttcgccgcccgccacggccgcatccccggcggcgcctgggtggtggtggcgatgggcaagctaggcagccggcagctcaccatcacctccgacatcgacctgatcgtggtctacgaggtgccgccgggcacccgccagtcggacggagccaagccgctggcccccaacgagtattacatcaagctgacgcagcgcctgaccaacgccatcaccgccccgatggccgacgggcggctgtacgaggtggacatgcggctgcgcccgtcgggcaacgccggtccgctcgccaccgcgctggacgccttcaccgcctatcaggccaaggatgcctggacgtgggagcacatggccctgacccgcgcccgcgtcatcggcagtgattccggcggcggcgatccggcgctgggtcataaggtcgagtcggcgatccgcggtgtgctgaccggcccgcgcgacccggccaaggtgctgcgggacgtcgccgacatgcgccgccgcatcgacaaggagttcggcaccaccaacccgtggaacgtcaaatacgcccgcggcggcctgatcgacatcgagttcacggcccagtatctccagctccgtcacggccacgcgcatccggacatcctgtccatcgccaccagccgcgccctgctcaacgccgctgcggccgggctgctggcgccggaggtggcggaggagctggtggcgacgctgaagctgtggcggcgggtgcagggcttcctgcgcctgaccaccgacggcgtgctcgatccgcggcaggtttcgcccaccctgcgggagggcctctcgcgcgccgccttcccggacgaggagccggcggttgacttcgccgcgctcgacagcagaatccgggacatcgccgcccgcgcccaccgccatttcgtggcgctggtcgaggagccggcgtcaaggctgcctcccccagagaccaacgaagaagccaaactcccatga

SEQ ID NO: 6 - DNA, 1500 bp, Azospirillum brasilense
  source 1..1500: mol_type = other DNA
atgcaccgcgggcgctaccgctccacccgctcgggccgcgcgcgggagctgctgaccgagctggtgccggccatgctgaacgagctggcgaagacccccgccccggacgacgcgctggttaagttcgacagcttcctggagcggcttccggcgggggtcgggctgttctcgctgttcatcgccaacccctggctgctcgccctggtcgcggagatcatgggcacggcgccgcagctggccgagacgttgtcgcgcaacccgtcgctgctcgacgccgtgctgtcgcccgacttcttcgacccgctgcccgacgcggcggggctgacgccggagtaccagcgcttcatcgccggggcgcacaacttcgaggatgtgctgaccctgtcgcggcgctggaccaacgaccagcgcttccgcgccggggcgcacatcctgcgcggcatcaccgacggcgaccgctgcggccccttcctcgccgatctggccgacgtggtggtgccggagctggccgcccgcgtggaggaggagttcgccgcccgccacggccgcatccccggcggcgcctgggtggtggtggcgatgggcaagctaggcagccggcagctcaccatcacctccgacatcgacctgatcgtggtctacgaggtgccgccgggcacccgccagtcggacggagccaagccgctggcccccaacgagtattacatcaagctgacgcagcgcctgaccaacgccatcaccgccccgatggccgatgggcggctgtacgaggtggacatgcggctgcgcccgtcgggcaacgccggtccgctcgccaccgcgctggacgccttcaccgcctatcaggccaaggatgcctggacgtgggagcacatggccctgacccgcgcccgcgtcatcggcagtgattccggcggcggcgatccggcgctgggtcataaggtcgagtcggcgatccgcggtgtgctgaccggcccgcgcgacccggccaaggtgctgcgggacgtcgccgacatgcgccgccgcatcgacaaggagttcggcaccaccaacccgtggaacgtcaaatacgcccgcggcggcctgatcgacatcgagttcacggcccagtatctccagctccgtcacggccacgcgcatccggacatcctgtccatcgccaccagccgcgccctgctcaacgccgctgcggccgggctgctggcgccggaggtggcggaggagctggtggcgacgctgaagctgtggcggcgggtgcagggcttcctgcgcctgaccaccgacggcgtgctcgatccgcggcaggtttcgcccaccctgcgggagggcctctcgcgcgccgccttcccggacgaggagccggcggttgacttcgccgcgctcgacagcagaatccgggacatcgccgcccgcgcccaccgccatttcgtggcgctggtcgaggagccggcgtcaaggctgcctcccccagagaccaacgaagaagccaaactcccatga

SEQ ID NO: 7 - DNA, 1428 bp, Azospirillum brasilense
  source 1..1428: mol_type = other DNA
atgctgaacgagctggcgaagacccccgccccggacgacgcgctggttaagttcgacagcttcctggagcggcttccggcgggggtcgggctgttctcgctgttcatcgccaacccctggctgctcgccctggtcgcggagatcatgggcacggcgccgcagctggccgagacgttgtcgcgcaacccgtcgctgctcgacgccgtgctgtcgcccgacttcttcgacccgctgcccgacgcggcggggctgacgccggagtaccagcgcttcatcgccggggcgcacaacttcgaggatgtgctgaccctgtcgcggcgctggaccaacgaccagcgcttccgcgccggggcgcacatcctgcgcggcatcaccgacggcgaccgctgcggccccttcctcgccgatctggccgacgtggtggtgccggagctggccgcccgcgtggaggaggagttcgccgcccgccacggccgcatccccggcggcgcctgggtggtggtggcgatgggcaagctaggcagccggcagctcaccatcacctccgacatcgacctgatcgtggtctacgaggtgccgccgggcacccgccagtcggacggagccaagccgctggcccccaacgagtattacatcaagctgacgcagcgcctgaccaacgccatcaccgccccgatggccgacgggcggctgtacgaggtggacatgcggctgcgcccgtcgggcaacgccggtccgctcgccaccgcgctggacgccttcaccgcctatcaggccaaggatgcctggacgtgggagcacatggccctgacccgcgcccgcgtcatcggcagtgattccggcggcggcgatccggcgctgggtcataaggtcgagtcggcgatccgcggtgtgctgaccggcccgcgcgacccggccaaggtgctgcgggacgtcgccgacatgcgccgccgcatcgacaaggagttcggcaccaccaacccgtggaacgtcaaatacgcccgcggcggcctgatcgacatcgagttcacggcccagtatctccagctccgtcacggccacgcgcatccggacatcctgtccatcgccaccagccgcgccctgctcaacgccgctgcggccgggctgctggcgccggaggtggcggaggagctggtggcgacgctgaagctgtggcggcgggtgcagggcttcctgcgcctgaccaccgacggcgtgctcgatccgcggcaggtttcgcccaccctgcgggagggcctctcgcgcgccgccttcccggacgaggagccggcggttgacttcgccgcgctcgacagcagaatccgggacatcgccgcccgcgcccaccgccatttcgtggcgctggtcgaggagccggcgtcaaggctgcctcccccagagaccaacgaagaagccaaactcccatga

SEQ ID NO: 8 - DNA, 1578 bp, synthetic construct
  misc_feature 1..1578: note = Codon-modified AT-C from E. coli
  source 1..1578: mol_type = other DNA
atggcgcacatgacgaacgtcaggagagtcttcaacgagctcatcggggatgacgagtcggagacgcaagaggaaagcttaagcgagcaatggagggaattatggcaagacgcactccaagaggacgataccacccccgtcttagcccacttgtcggaagacgacaggaagcaagttttgaccttaatcgcggactttaggaaggaattagacaaaaggacgattggccccaggggcaggcaagtcttggatcacttaatgccccacttattgtccgacgtgtgcgccagggaggatgccgcagtgaccttaagcagaatcacggcgctcttagtcggcatcgtcacgaggacgacgtacctggagctcttgtcggagtttccggccgccttgaagcacctcatcagcttatgcgcggccagccccatgatcgcgtcgcaattagccagatacccgctgcttttagacgagctcttggacccgaatacgttgtatcaacccaccgccacagacgcgtatagggacgaactcagacaataccttttaagagtccccgaggacgatgaggaacagcaattggaagccttaaggcaatttaagcaagcccaattactgagaattgcagccgcagacattgcgggcaccttgcccgttatgaaggtttcggaccatctgacgtggttagccgaggcgatgatcgacgcggtggtccaacaagcctgggtccagatggtcgcgaggtatggcaaaccaaaccatttaaatgagagagagggcagaggcttcgcagtcgtgggatatgggaaattaggggggtgggaactggggtattccagtgacttggatttgatatttttgcacgactgtcccatggacgccatgacggatggcgaaagagagattgatggaagacaattctacttaaggttagcccagaggatcatgcacttattttccaccaggacgagcagtgggatcctctacgaggtcgacgccaggttaagacctagtggcgccgccggcatgttagtcaccagtgctgaggcgttcgcggactaccaaaagaatgaagcatggacctgggagcaccaagccttagttagggccagggtggtctatggagacccccaattgacggcccatttcgatgcggtcagaagggaaatcatgaccttacccagggagggcaagaccttacagaccgaggtcagagagatgagagaaaagatgagggcccacttggggaacaagcacagagacaggttcgacataaaggcggacgagggcggcatcacggacattgagttcatcactcagtacttagtcctcagatatgcccacgagaagcccaaactgaccagatggagtgataatgtcagaatcttagagttgttagctcagaatgatatcatggaggaacaagaggccatggccttaacgagggcctatacgaccttgagggacgagttgcaccacttagcgctgcaagagctccccgggcacgtcagcgaagactgttttacggccgaaagagagttagttagagcgtcttggcaaaaatggttagtcgaggagtga

SEQ ID NO: 9 - DNA, 1029 bp, p1 bacteriophage
  source 1..1029: mol_type = other DNA
gtgaatttactgaccgtacaccaaaatttgcctgcattgccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcgtatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgactaa

SEQ ID NO: 10 - DNA, 34 bp, synthetic construct
  misc_feature 1..34: note = loxp site
  source 1..34: mol_type = other DNA
ataacttcgtataatgtatgctatacgaagttat

(19) United States
(12) Patent Application Publication
(10) Pub. No.: US 2026/0242802 A1
(43) Pub. Date: Aug. 20, 2026

(54) Inducible Ammonia Production from a Symbiotic Diazotroph, Methods of Creation and Uses Thereof

(71) Applicant: The Board of Trustees of the Leland Stanford Junior University, Stanford, CA (US)

(72) Inventors: Tim Schnabel, Stanford, CA (US); Elizabeth Sattely, Stanford, CA (US)

(73) Assignee: The Board of Trustees of the Leland Stanford Junior University, Stanford, CA (US)

(21) Appl. No.: 19/354,751

(22) Filed: Oct. 9, 2025

Related U.S. Application Data
(62) Division of application No. 17/428,927, filed on Feb. 5, 2020, now Pat. No. 12,460,213.
(60) Provisional application No. 62/801,454, filed on Feb. 5, 2019.

(51) Int. Cl. C12N 15/63; A01N 63/20; C12N 9/12; C12N 15/74; C12P 3/00

(52) U.S. Cl. CPC C12N 15/635; A01N 63/20; C12N 9/1241; C12N 15/74; C12P 3/00; C12Y 207/07051

(57) ABSTRACT

The present disclosure describes systems and methods capable of fixing atmospheric nitrogen into bio-available nitrogenous compounds, including ammonia. Embodiments of the present disclosure are directed to synthetic DNA constructs encoding genes to allow release of bio-available nitrogenous compounds in nitrogen fixing diazotrophic organisms. Many of these constructs encode these genes in inducible and constitutive means, such that inducible embodiments can be activated at select times. Additional embodiments are directed to genetically engineered diazotrophs utilizing these constructs to produce bio-available nitrogenous compounds. Further embodiments are directed to methods to create these constructs and organisms as well as to use these constructs and organisms.

### CROSS REFERENCE TO RELATED APPLICATIONS

This application is a divisional of U.S. application Ser. No. 17/428,927, which is the National Stage of PCT Patent Appl. No. PCT/US2020/016856 filed Feb. 5, 2020, which claims benefit of U.S. Provisional Appl. No. 62/801,454 filed Feb. 5, 2019, the disclosures of which are hereby incorporated by reference in their entireties.

### SEQUENCE LISTING

This application hereby incorporates by reference the material of the electronic Sequence Listing filed concurrently herewith. The material in the electronic Sequence Listing is submitted as an ST26 XML (.xml) file entitled “S31-05977” created on Oct. 9, 2025, having a file size of 22,800 bytes and is hereby incorporated by reference in its entirety. The XML file is identical to the ST25 TXT file of the parent application. No new matter is added by the ST26 sequence listing file.

### FIELD OF THE INVENTION

The present invention relates generally to in vivo nitrogen fixation in genetically modified organisms, including methods to create nitrogen fixing organisms and uses thereof, more particularly, bacteria genetically engineered to release nitrogenous compounds that are available for biological use. The present invention is also directed to formulations and methods of using genetically engineered nitrogen fixing organisms.

### BACKGROUND OF THE INVENTION

Nitrogen is the major limiting nutrient on industrially farmed land. While the atmosphere is comprised of approximately 78% nitrogen (N₂), a strong triple bond between the nitrogen atoms makes N₂ inert and inaccessible to some species. Current methods of agriculture have depleted organic soil nitrogen faster than it can be replenished naturally. (See e.g., Developments in Soil Science, Vol. 3. (ed. F. E. Allison) 120-138 (Elsevier, 1973); the disclosure of which is incorporated herein in its entirety.) Converting nitrogen from N₂ to a bio-available form such as ammonia (NH₃) is called nitrogen fixation. As fields are depleted of organic nitrogen reserves, over 100 million tons per year of synthetically fixed nitrogen fertilizer currently support crops feeding approximately 3 billion people. (See e.g., Davidson, E. A. et al. Excess nitrogen in the US Environment: trends, risks and solutions. ESA Issue Ecol 15 (2012); and Galloway, J. N. et al. Transformation of the Nitrogen Cycle: Recent Trends, Questions, and Potential Solutions. Science 320, 889 (2008); the disclosures of which are incorporated herein in their entireties.) Furthermore, the Haber-Bosch process, which currently fixes the vast majority of nitrogen used for fertilizer, is unsustainable because it is energy intensive, produces potent greenhouse gasses, and generates fertilizer for bulk application, which leads to inefficient plant uptake, microbial scavenging, NxOx pollution, leaching and eutrophication, and water poisoning. Additionally, nitrogen fixation through the Haber-Bosch process is highly centralized and does not reach many parts of the world where farmers are dependent on poor quality soils.

A highly sought-after solution is the biological provision of fixed nitrogen directly at the root of plants. (See e.g., Pankievicz, V. C. S., et al. Are we there yet? The long walk towards the development of efficient symbiotic associations between nitrogen-fixing bacteria and non-leguminous crops. BMC Biology 17, 99 (2019); Mus, F. et al. Symbiotic Nitrogen Fixation and Challenges to Extending it to Non-Legumes. Applied and Environmental Microbiology (2016); and Wurtzel, E. T. et al. Revolutionizing agriculture with synthetic biology. Nature Plants 5, 1207-1210 (2019); the disclosures of which are incorporated herein in their entireties.) Across several phyla, about 15% of prokaryotes have evolved diazotrophy: the capability to convert nitrogen gas (N₂) to bioavailable ammonia through an enzymatic nitrogen fixation process. (See e.g., Dos Santos, P. C., et al. Distribution of nitrogen fixation and nitrogenase-like sequences amongst microbial genomes. BMC Genomics 13, 162 (2012); the disclosure of which is incorporated herein in its entirety.) Some plants have evolved intimate symbioses with a subset of these diazotrophs to obtain bioavailable nitrogen, primarily limited to nodulating species of the Fabaceae family of plants and diazotrophic Rhizobia. (See e.g., Long, S. R. Genes and Signals in the Rhizobium-Legume Symbiosis. Plant Physiology 125, 69 (2001); the disclosure of which is incorporated herein in its entirety.) However, these plants do not include most domesticated crops: 50% of the world's calories come from the cereal grasses, including corn, rice, and the grains, that are not known to participate in tight nitrogen transfer symbioses. (See e.g., Mus, F. et al. Symbiotic Nitrogen Fixation and Challenges to Extending it to Non-Legumes. Applied and Environmental Microbiology (2016); Awika, J. M. in Advances in Cereal Science: Implications to Food Processing and Health Promotion, Vol. 1089 1-13 (American Chemical Society, 2011); and Rogers, C. & Oldroyd, G. E. D. Synthetic biology approaches to engineering the nitrogen symbiosis in cereals. J Exp Bot 65 (2014); the disclosures of which are incorporated herein in their entireties.) Outside the Rhizobia, many bacterial diazotrophs colonize plant roots through looser association in the rhizosphere or endophytic compartments. Associative diazotrophs from several genera, including Azospirillum, Herbaspirillum, Gluconacetobacter, and Burkholderia have been shown to promote plant growth and contribute to plant nitrogen supply, however, there is still a big gap between what plants require and what they currently obtain. (See e.g., Rosenblueth, M. et al. Nitrogen Fixation in Cereals. Frontiers in Microbiology 9, 1794 (2018); Pankievicz, V. C. S. et al. Robust biological nitrogen fixation in a model grass-bacterial association. The Plant Journal 81, 907-919 (2015); Santos, K. F. D. N. et al. Wheat colonization by an Azospirillum brasilense ammonium-excreting strain reveals upregulation of nitrogenase and superior plant growth promotion. Plant and Soil 415, 245-255 (2017); Christiansen-Weniger, C. & van Veen, J. A. Nitrogen fixation by Azospirillum brasilense in soil and the rhizosphere under controlled environmental conditions. Biology and Fertility of Soils 12, 100-106 (1991); Boddey, R. M., et al. Endophytic nitrogen fixation in sugarcane: present knowledge and future applications. Plant and Soil 252, 139-149 (2003); and Divan Baldani, V. L., et al. Inoculation of rice plants with the endophytic diazotrophs Herbaspirillum seropedicae and Burkholderia spp. Biology and Fertility of Soils 30, 485-491 (2000); the disclosures of which are incorporated herein in their entireties.)

Prior engineering efforts have focused on associative diazotrophs to address the shortcoming in biological nitrogen provision for non-nodulating crops. One target has been the nitrogen fixation step directly, while a second target has been the nitrogen assimilation pathway, centered around glutamine synthetase (GS, gene abbreviation: g InA).

The former strategy involves the heterohexamer nitrogenase NifHDK complex and its many constituents that are required for its activation and efficient reduction of nitrogen to ammonia. Optimizing the NifHDK machinery for higher nitrogen fixation rates, for fixed nitrogen release, or for nitrogen fixation in heterologous organisms including plants, remains a challenge due to complex multilayered regulation at the transcriptional, translational and posttranslational levels. (See e.g., Temme, K., et al. Refactoring the nitrogen fixation gene cluster from Klebsiella oxytoca. Proc Natl Acad Sci USA 109, 7085-7090 (2012); Dixon, R. & Kahn, D. Genetic regulation of biological nitrogen fixation. Nature Reviews Microbiology 2, 621-631 (2004); and Curatti, L. & Rubio, L. M. Challenges to develop nitrogen-fixing cereals by direct nif-gene transfer. Plant Science 225, 130-137 (2014); the disclosures of which are incorporated herein in their entireties.)

Regarding the second strategy—the nitrogen assimilation pathway: Prior methods have focused on deregulating GS via active site mutation and/or inclusion of inducible promoters without direct interference in the complex regulatory network surrounding NifHDK. (See e.g., Ortiz-Marquez, J. C., et al. Metabolic engineering of ammonium release for nitrogen-fixing multispecies microbial cell-factories. Metab Eng 23, 154-164 (2014); Ambrosio, R., et al. Metabolic engineering of a diazotrophic bacterium improves ammonium release and biofertilization of plants and microalgae. Metabolic Engineering 40, 59-68 (2017); Wu, N. et al. Alpha-Ketoglutarate: Physiological Functions and Applications. Biomolecules & therapeutics 24, 1-8 (2016); and Jiang, P., et al. Structure-function analysis of glutamine synthetase adenylyltransferase (ATase, EC 2.7.7.49) of Escherichia coli. Biochemistry 46, 4117-4132 (2007); the disclosures of which are incorporated herein in their entireties.) However, these methods risk additional consequences, as GS is polycistronic and transcriptionally co-regulated with other critical regulatory genes, such as gInB in Azospirillum brasilense and Gluconacetobacter diazotrophicus, and ntrB and ntrC in Herbaspirillum seropedicae. (See e.g., de Zamaroczy, M., et al. Functional organization of the glnB-glnA cluster of Azospirillum brasilense. Journal of Bacteriology 175, 2507-2515 (1993); and Schwab, S. et al. The glnAntrBC operon of Herbaspirillum seropedicae is transcribed by two oppositely regulated promoters upstream of ginA. Canadian Journal of Microbiology 53, 100-105 (2007); the disclosures of which are incorporated herein in their entireties.)

### SUMMARY OF THE INVENTION

Systems and methods for engineering diazotrophs to release nitrogen containing compounds in accordance with embodiments of the invention are disclosed.

In one embodiment, an inducible genetic circuit to deactivate glutamine synthetase in an organism includes a unidirectional adenylyl transferase capable of adenylylating glutamine synthetase and a system for inducing expression of the unidirectional adenylyl transferase enzyme.

In a further embodiment, the unidirectional adenylyl transferase is selected from the SEQ ID NOs: 1-7.

In another embodiment, the unidirectional adenylyl transferase is SEQ ID NO: 6.

In a still further embodiment, the system for inducing expression is a tetracycline inducing system.

In still another embodiment, the inducible genetic circuit further includes a second unidirectional adenylyl transferase capable of adenylylating glutamine synthetase.

In a yet further embodiment, the unidirectional adenylyl transferase and the second unidirectional adenylyl transferase are polycistronic.

In yet another embodiment, the unidirectional adenylyl transferase and the second unidirectional adenylyl transferase have separate promoters.

In a further embodiment again, an engineered diazotroph to release nitrogenous compounds into the environment includes a diazatrophic organism and a genetic circuit to allow expression of an enzyme capable of deactivating glutamine synthetase within the diazatrophic organism, wherein the genetic circuit is integrated into chromosomal DNA of the diazatrophic organism.

In another embodiment again, the genetic circuit includes a unidirectional adenylyl transferase capable of adenylylating glutamine synthetase and a system for inducing expression of the unidirectional adenylyl transferase enzyme.

In a further additional embodiment, the unidirectional adenylyl transferase is selected from the SEQ ID NOs: 1-7.

In another additional embodiment, the unidirectional adenylyl transferase is SEQ ID NO: 6.

In a still yet further embodiment, the system for inducing expression is a tetracycline inducing system.

In still yet another embodiment, the genetic circuit further includes a second unidirectional adenylyl transferase capable of adenylylating glutamine synthetase.

In a still further embodiment again, the unidirectional adenylyl transferase and the second unidirectional adenylyl transferase are polycistronic.

In still another embodiment again, the unidirectional adenylyl transferase and the second unidirectional adenylyl transferase have separate promoters.

In a still further additional embodiment, a method of increasing bio-available nitrogen in soil includes obtaining an engineered diazotrophic organism, wherein the engineered diazotrophic organism contains a genetic circuit capable of deactivating glutamine synthetase within the diazatrophic organism and introducing the engineered diazotrophic organism into an environment containing soil.

In still another additional embodiment, the diazotrophic organism comprises a genetic circuit to allow expression of an enzyme capable of deactivating glutamine synthetase within the diazatrophic organism, wherein the genetic circuit is integrated into chromosomal DNA of the diazatrophic organism.

In a yet further embodiment again, the genetic circuit includes a unidirectional adenylyl transferase capable of adenylylating glutamine synthetase and a system for inducing expression of the unidirectional adenylyl transferase enzyme.

In yet another embodiment again, the unidirectional adenylyl transferase is selected from the SEQ ID NOs: 1-7.

In a yet further additional embodiment, the unidirectional adenylyl transferase is SEQ ID NO: 6.

In yet another additional embodiment, the system for inducing expression is a tetracycline inducing system.

In a further additional embodiment again, the genetic circuit further comprises a second unidirectional adenylyl transferase capable of adenylylating glutamine synthetase.

In another additional embodiment again, the unidirectional adenylyl transferase and the second unidirectional adenylyl transferase are polycistronic.

In a still yet further embodiment again, the unidirectional adenylyl transferase and the second unidirectional adenylyl transferase have separate promoters.

In still yet another embodiment again, the method further includes applying an inducer to the soil.

In a still yet further additional embodiment, the inducer is tetracycline.

Additional embodiments and features are set forth in part in the description that follows, and in part will become apparent to those skilled in the art upon examination of the specification or may be learned by the practice of the disclosure. A further understanding of the nature and advantages of the present disclosure may be realized by reference to the remaining portions of the specification and the drawings, which forms a part of this disclosure.

### BRIEF DESCRIPTION OF THE DRAWINGS

These and other features and advantages of the present invention will be better understood by reference to the following detailed description when considered in conjunction with the accompanying drawings where:

FIG. 1A illustrates a schematic of glutamine synthetase regulation in accordance with various embodiments of the invention.

FIG. 1B illustrates a schematic of engineered adenylyl transferases (ATases) in accordance with various embodiments of the invention.

FIG. 2 illustrates exemplary unidirectional ATases (uATs) in accordance with various embodiments of the invention.

FIGS. 3A-3G illustrate exemplary genetic expression circuits in accordance with various embodiments of the invention.

FIGS. 4A-4B illustrate exemplary plasmids for genetic transformation in accordance with various embodiments of the invention.

FIG. 5A illustrates ammonia production levels of exemplary embodiments of uATs in accordance with various embodiments of the invention.

FIG. 5B illustrates ammonia production rates and glutamine synthetase adenylylation states of exemplary embodiments of uATs in accordance with various embodiments of the invention.

FIGS. 6A-6B illustrate incorporation of nitrogen gas (N₂) after inoculation of diazotrophs with uATs in accordance with various embodiments of the invention.

FIGS. 7A-7C illustrate ammonia production rates of exemplary embodiments of uATs in accordance with various embodiments of the invention.

FIGS. 8A-8C illustrate plant growth and health indicators of plants after growth with engineered diazotrophs in accordance with various embodiments of the invention.

FIG. 8D illustrates incorporation of N₂ into pheophytin in accordance with various embodiments of the invention.

FIG. 9A illustrates an exemplary genetic expression circuit in accordance with various embodiments of the invention.

FIG. 9B illustrates ammonia production levels of exemplary embodiments of uATs and CreRs in accordance with various embodiments of the invention.

FIG. 10 illustrates a flow chart of a method of increasing bio-available nitrogen in soil in accordance with various embodiments of the invention.

### DETAILED DESCRIPTION

The embodiments of the invention described herein are not intended to be exhaustive or to limit the invention to precise forms disclosed. Rather, the embodiments selected for description have been chosen to enable one skilled in the art to practice the invention.

Turning now to the drawings, systems, methods of making, and methods of using inducible symbiotic diazotrophs are illustrated. Many embodiments are directed to novel genes capable of post-transcriptionally regulating glutamine synthetase (GS) by adenylylating GS via a GS adenylyltransferase (ATase), including g/nE. A number of embodiments create a unidirectional ATase that is capable of adenylylating GS and not capable of removing (e.g., hydrolyzing) the adenylyl group from GS. Additional embodiments incorporate novel genes into genetic circuits to constitutively or inducibly express these genes in vivo. Further embodiments are directed to diazotrophic organisms capable of producing bio-available nitrogen, including ammonia. Certain embodiments produce ammonia by regulating glutamine synthetase (GS).

Glutamine synthetase (GS) is a ubiquitous enzyme that combines ammonia and glutamate to synthesize glutamine. GS activity is required for cellular nitrogen cycling, since it is the primary route by which ammonia initially enters or re-enters the highly branched cellular metabolism of nucleic acid, amino acid, and secondary metabolite biosynthesis. (See e.g., Stadtman, E. R. Regulation of Glutamine Synthetase Activity. EcoSal Plus 1 (2004); and Merrick, M. J. & Edwards, R. A. Nitrogen control in bacteria. Microbiol Rev 59, 604-622 (1995); the disclosures of which are incorporated herein in their entireties.)

Because transcriptional regulation of GS has many consequences, non-transcriptional regulation to regulate enzyme activity may be a better target for producing ammonia by deactivating GS. ATases, including g/nE, post-translationally deactivates GS through adenylylation. This process is reversible by hydrolysis of the adenylyl groups by the same enzyme. Previous work has shown that in Escherichia coli the adenylyltransferring and the adenylylremoving activities reside in separate, homologous protein domains and that these can function independently to modulate GS activity in vitro. 33, 36. (See e.g., Jiang, P., et al. Structure-function analysis of glutamine synthetase adenylyltransferase (ATase, EC 2.7.7.49) of Escherichia coli. Biochemistry 46, 4117-4132 (2007); and Jaggi, R., et al. The two opposing activities of adenylyl transferase reside in distinct homologous domains, with intramolecular signal transduction. Embo j 16, 5562-5571 (1997); the disclosures of which are incorporated herein in their entireties.)

### Unidirectional ATases

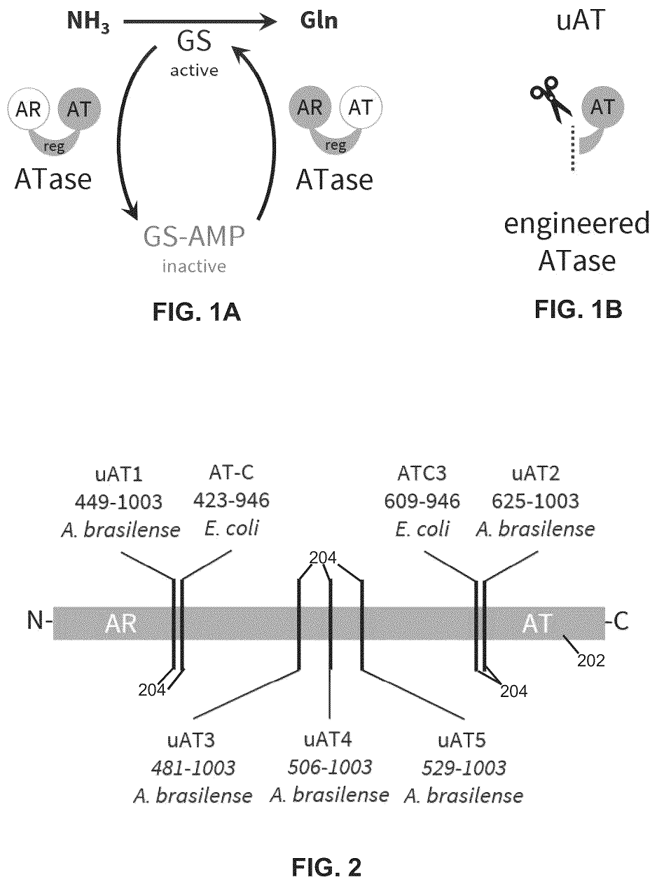

Turning to FIG. 1A, a schematic of the regulation of glutamine synthetase (GS) is illustrated. In particular, active GS is responsible for catalyzing the reaction of ammonia (NH₃) and glutamate to glutamine (Gin). However, when GS is adenylylated (GS-AMP), activity of GS is shut off, and glutamine is not produced by a cell and/or an organism. The adenylylation of GS is performed by an adenylyl transferase (ATase). However, many ATases possess adenylyl transfer domain (AT) and adenylyl removal domain (AR), thus allowing ATases to both activate and deactivate GS. Turning to FIG. 1B, many embodiments will engineer ATases to possess only the AT domains, thus becoming a unidirectional ATase (uAT) to deactivate GS.

A number of ways exist in the art to generate unidirectional ATases capable of adenylylating GS. For example, certain embodiments will delete the native ATase gene (e.g. glnE) and then introduce uATs. Further embodiments will introduce point mutations within an ATase that disrupt activity in the AR domain. Additional embodiments will manipulate genes to produce truncated ATases that are missing some or all of the AR domain. To identify ATase genes and domains within these genes, a number of embodiments will align gene and/or protein (known or predicted) sequences to identify AT domains and ATase genes. Turning to FIG. 2, an example of a native ATase peptide 202 is illustrated along with truncation sites in accordance with certain embodiments. In particular, the ATase peptide 202 spans from the N-terminus (N) to the C-terminus (C), with the AR domain existing proximal the N-terminus, and the AT domain existing proximal to the C-terminus. Truncation sites 204 represent exemplary uATs of certain embodiments, where the mature ATase enzyme from E. coli or A. brasilense have been truncated to the amino acids noted in FIG. 2. The uATs illustrated in FIG. 2 are summarized in Table 1 along with sequence identification numbers (SEQ ID NOs) for their respective coding sequences. It should be noted that while uATs generated from E. coli and A. brasilense are provided herein, many embodiments are not limited to these species, and further embodiments will create uATs from additional species, including many diazotrophic organisms. Additional embodiments will further truncate an ATase at additional locations. Diazotrophic organisms in accordance with a number of embodiments include Proteobacteria (such as Pseudomonas, Enterobacter, Stenotrophomonas, Burkholderia, Rhizobium, Herbaspirillum, Pantoea, Serratia, Rahnella, Azospirillum, Azorhizobium, Azotobacter, Duganella, Delftia, Bradyrhizobium, Sinorhizobium, and Halomonas), Firmicutes (such as Bacillus, Paenibacillus, Lactobacillus, Mycoplasma, and Acetobacterium), and Actinobacteria (such as Streptomyces, Rhodococcus, Microbacterium, and Curtobacterium).

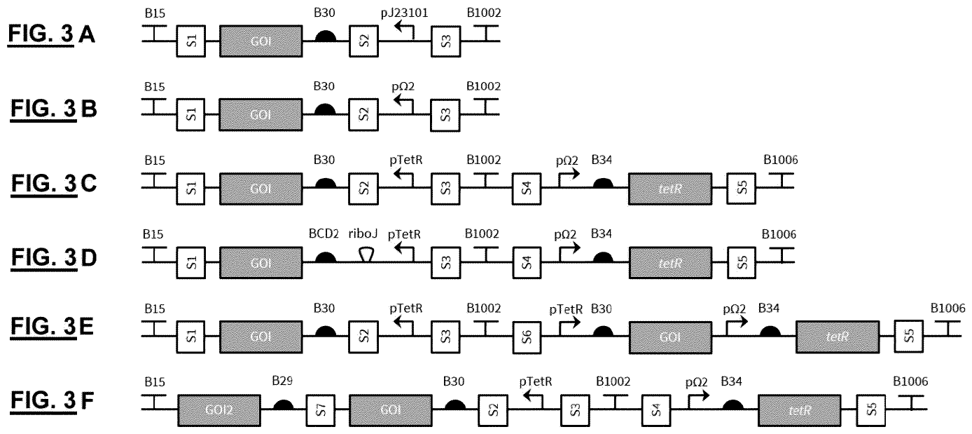

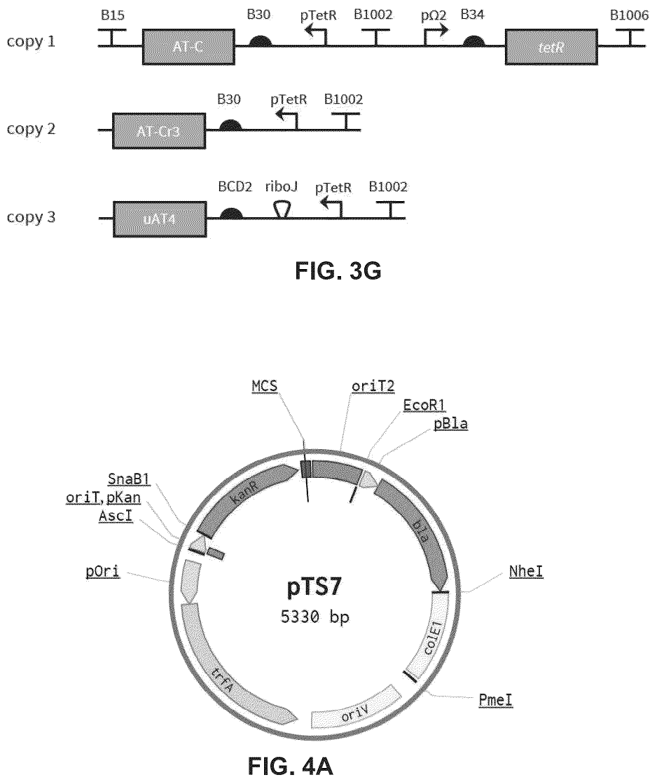

A number of embodiments will integrate uATs into a genetic circuit including promoter systems for constitutive or inducible expression. Turning to FIGS. 3A-3G, exemplary circuits are illustrated, such that a gene of interest (GOI) (e.g., uAT) is activated either constitutively or inducibly. Specifically, FIGS. 3A-3B illustrate constitutive expression circuits to cause embodiments to produce uAT without exogenous control, while FIGS. 3C-3G illustrate inducible circuits, where expression of the GOI is induced by an exogenous inducer. Certain embodiments will use a tetracycline inducing system, such that exposure to tetracycline will allow expression of the GOI (e.g., uAT). While FIGS. 3C-3G illustrate systems inducible using tetracycline, further inducible circuits can be utilized in various embodiments, including systems that induce expression using quorum sensing molecules and/or plant produced molecules. Further, certain embodiments will use a repressor, such that a GOI is repressed in the presence of a molecule, but it activates in the absence of the molecule. Further embodiments will utilize a system that allows induction and repression of a GOI at will by addition of certain molecules. Still further embodiments will utilize an oscillatory circuit to regulate GS activity, where oscillatory circuits allow for the genetic to circuit to oscillate between “on” and “off” states of expression at certain intervals, such as time or cellular reproduction. Many circuits in accordance with various embodiments further possess spacer sequences (e.g., S1-S4), enhancer sequences, and/or any additional type sequence known in the art to increase, enhance, and/or enable gene expression.

Additionally, FIGS. 3E-3G illustrate systems to express more than one gene of interest via an inducible system-specifically, FIG. 3E illustrates a separate promoter for each GOI, while FIG. 3F illustrates a polycistronic system for expressing the multiple GOIs with a single promoter. In certain embodiments based on FIGS. 3E-3F, the multiple genes of interest are copies of the same gene (e.g., 2 copies of uAT), while certain embodiments will introduce single copies of multiple genes (e.g., 1 copy of uAT and 1 copy of another gene of interest). In many embodiments, additional genes of interest will be selected from genes that enhance ammonia production and/or release from a cell. Certain embodiments will include reporter genes (e.g., green fluorescent protein; GFP). A number of embodiments will select additional genes of interest, including: amtB, fdxB, fdxN, fixX, GFP, RFP, and/or YFP. FIG. 3G further illustrates embodiments possessing multiple copies of inducible uATs in accordance with many embodiments. Specifically, FIG. 3G illustrates an inducible system possessing three copies of uATs, where each uAT is a different variant of a uAT at a different chromosomal location. For example, copy 1 shows an example with the AT-C(SEQ ID NO: 1) uAT, while copy 2 shows AT-Cr3 (SEQ ID NO: 8), which is a sequence variant of AT-C that produces the same peptide as AT-C, but the sequence variation limits recombination between the two copies. Further, copy 3 possesses the uAT, uAT4 (SEQ ID NO: 4). By placing multiple uAT copies at different chromosomal locations, certain embodiments may prevent native machinery from recombining or excising (thus deactivating) all uAT copies simultaneously. The use of different uAT variants may provide additional stability between the copies and make the deactivation of these genes more difficult, which is further described herein, as deactivating multiple uATs is more difficult than deactivating a single uAT. It should be noted that while FIGS. 3E-3G illustrate embodiments with two GOIs, a number of embodiments will include 3 or more genes of interest in an expression system. Further embodiments will delete all but one copy of GS genes within a diazotroph's genome. With one remaining GS gene, this GS gene can be deactivated with at least one uAT transformed into the diazotroph.

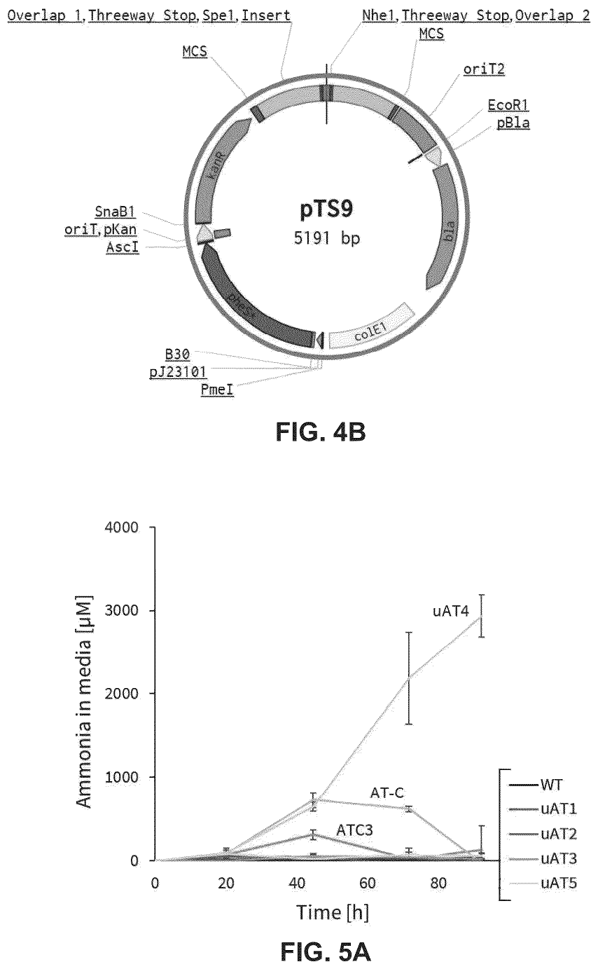

Many embodiments will be directed to engineered organisms that express one or more uATs, such that native GS will be deactivated. In these embodiments, deactivating native GS will block glutamine synthesis and allow release and/or production of ammonia from the organism. Various embodiments will place uATs into replicative plasmids, while additional embodiments will place uATs in integrative plasmids. FIGS. 4A-4B illustrate exemplary replicative (FIG. 4A) and integrative (FIG. 4B) plasmids in accordance with many embodiments. Integrative plasmids allow for integration of a genetic circuit into chromosomal DNA of a recipient diazotroph. Integration can either occur by single or double homologous recombination, for example FIG. 3G copies are integrated by double homologous recombination. A number of embodiments will deactivate the native ATase gene within the organism being engineered, such that the native ATase will not reactivate GS via hydrolysis of an adenylyl group. One of skill in the art will understand methods to deactivate innate ATases, including via deletion, mutation, and/or any other suitable method to remove innate ATase activity. Once uATs are placed into plasmids (replicative or integrative), diazotrophs of a number of embodiments will be transformed with these plasmids. One of skill in the art will understand a number of methodologies for introducing plasmids into diazotrophs, including one or more of the following: electroporation, particle bombardment, mediated transformation (e.g., viral vector or transposase driven), conjugation (e.g., with donor or donor and helper conjugation systems), heat shock, and/or any other method that will yield stable transformation of uATs into a diazotroph.

### Ammonia Production and GS Regulation

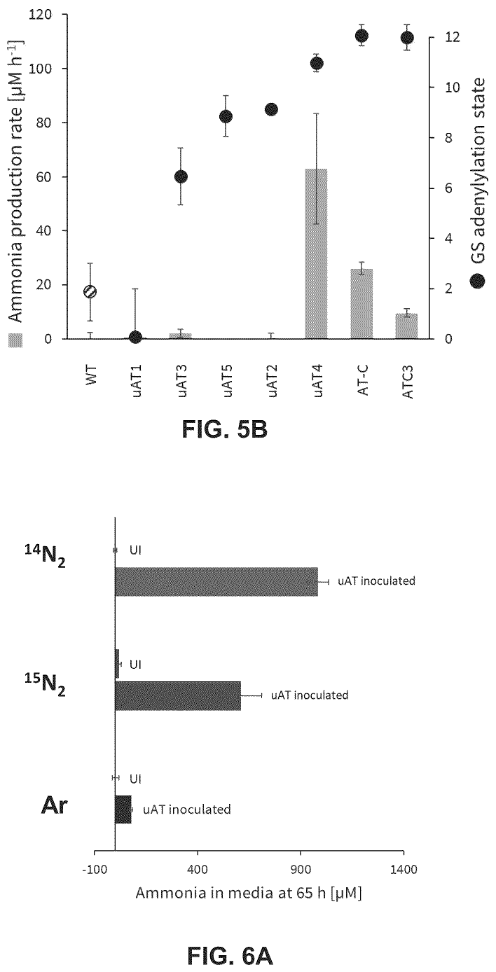

Many embodiments of diazotrophs will produce ammonia after transformation with uAT. Turning to FIG. 5A, ammonia production in a number of embodiments are illustrated. In particular, FIG. 5A illustrates that a number of embodiments are capable of producing ammonia in media at levels of approximately 0.3 mM to approximately 3 mM at certain inoculating ODs. Additionally, FIG. 5B illustrates the adenylylation state of GS and ammonia production in embodiments transformed with a number of uATs. In particular, these data show how a number of uATs increase the adenylylation state, leading to deactivation of GS as well as ammonia production in these embodiments. In A. brasilense, GS has a maximum adenylylation state of 12, representing the 12 possible adenylylation sites on the enzyme. In FIG. 5B, uATs with an adenylylation state of 8 or less did not produce ammonia in a significant quantity (e.g., uAT1, uAT2, uAT3, uAT5), indicating that many embodiments will be directed to uATs capable of creating a GS adenylylation state of 9 or greater.

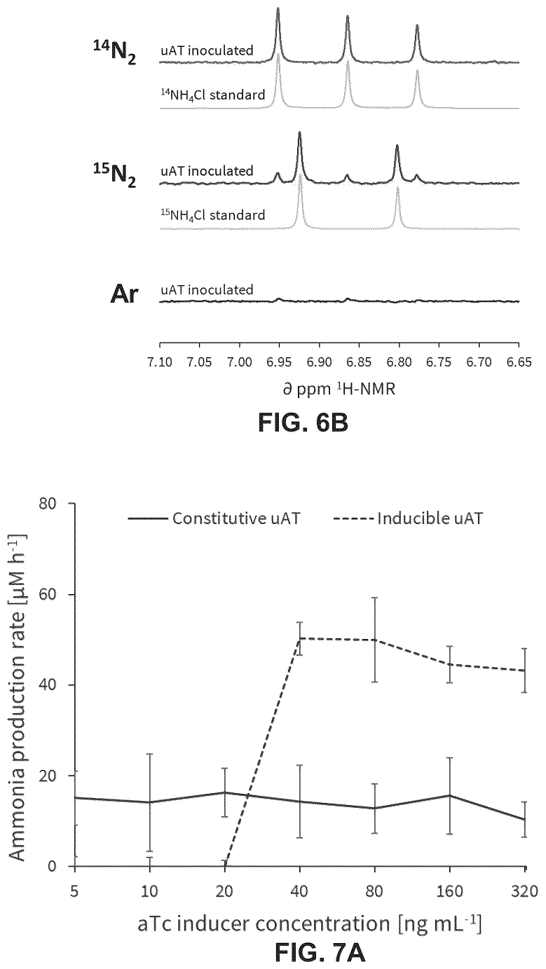

Turning to FIGS. 6A-6B, the ammonia production is due to nitrogen fixation from embodiments in controlled atmospheres. In particular, FIG. 6A illustrates ammonia production of uAT expressing diazotrophs inoculated into media in accordance with many embodiments as well as an uninoculated (UI) control in atmospheres of ¹⁴N₂, ¹⁵N₂, and Ar, showing that the introduction of a uAT expressing diazotroph of many embodiments leads to ammonia production. Further, FIG. 6B illustrates NMR results of the ammonia produced in the controlled environments, where the ammonia produced matches the isotopic nitrogen gas, thus the ammonia is coming from embodiments expressing a uAT and not an outside source.

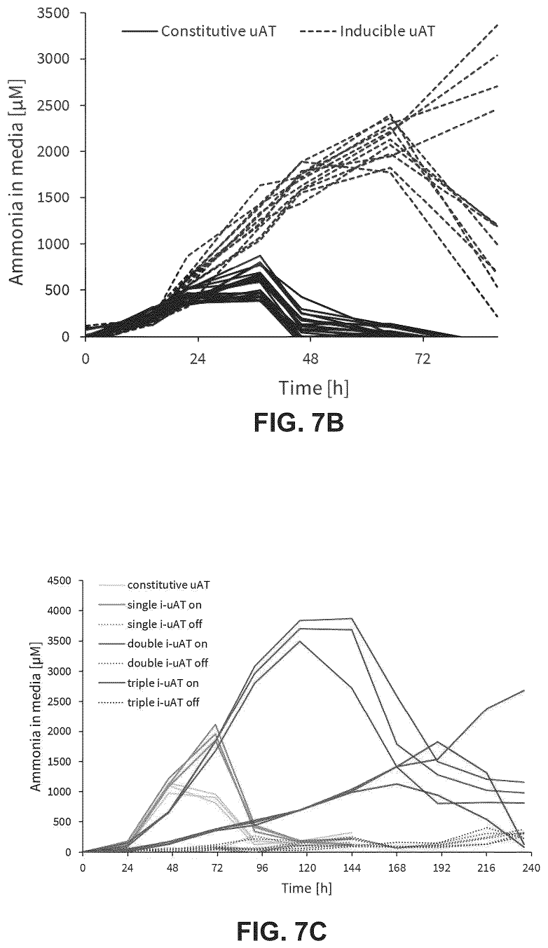

Turning to FIGS. 7A-7B, ammonia production in embodiments of constitutive and inducible uATs are illustrated. In FIGS. 7A-7B, a uAT in accordance with certain embodiments was placed in a constitutive expression system (e.g., FIGS. 3A-3B) and an inducible expression system (e.g., FIGS. 3C-3F). As an inducer concentration (x-axis) was increased, the inducible system embodiments (dashed line) were activated and produced ammonia at a rate of approximately 50 μM/hr at certain inoculating ODs, while constitutive embodiments produced maintained an approximately 18 μM/hr production rate at the same inoculating OD and independent of an inducer concentration. FIG. 7A illustrates that embodiments with inducible systems may be capable of producing ammonia at a higher rate than constitutive systems. Turning to FIG. 7B, embodiments with inducible uATs are capable of outlasting constitutive uAT embodiments. Specifically, the total ammonia in media concentration is plotted against time with a number of constitutive uAT embodiments (solid lines) and inducible uAT embodiments (dashed lines). The reduction of ammonia in media indicates that certain diazotrophs may compete against the uAT to return glutamine production from the produced ammonia. As shown in FIG. 7B, constitutive uAT embodiments begin to lose ammonia in concentration after approximately 36 hours, while several inducible systems produce ammonia until approximately the 72-hour mark, before the ammonia concentration begins to fall. It should be noted that a number of inducible uAT embodiments did not illustrate a reduction in ammonia within the timeframe of this figure.

In FIG. 7C, the ammonia concentration over time of embodiments with a constitutive uAT and embodiments of various copies of inducible uATs is illustrated. Specifically, FIG. 7C illustrates that a constitutively active uAT peaked at approximately 1 mM at approximately 48 hours, while embodiments with one inducible uAT copy peaks at approximately 2 mM at 72 hours, embodiments with two inducible uAT copies peaks at approximately 4 mM at 120 hours, and embodiments with three inducible uAT copies peaks at approximately 2 mM at 192 hours, indicating that multiple copies of uATs in many embodiments increases the stability of ammonia production.

A number of embodiments will transform uAT into one or more of the following diazotrophic organisms: Proteobacteria (such as Pseudomonas, Enterobacter, Stenotrophomonas, Burkholderia, Rhizobium, Herbaspirillum, Pantoea, Serratia, Rahnella, Azospirillum, Azorhizobium, Azotobacter, Duganella, Delftia, Bradyrhizobium, Sinorhizobium, and Halomonas), Firmicutes (such as Bacillus, Paenibacillus, Lactobacillus, Mycoplasma, and Acetobacterium), and Actinobacteria (such as Streptomyces, Rhodococcus, Microbacterium, and Curtobacterium. Different diazotrophic organisms may occupy or colonize different root niches. Certain embodiments will transform and apply a consortium of diazotrophic organisms. By applying a consortium of symbiotic diazotrophs, many embodiments can maximize the amount of fixed nitrogen supplied to a plant.

### Increasing Plant Growth

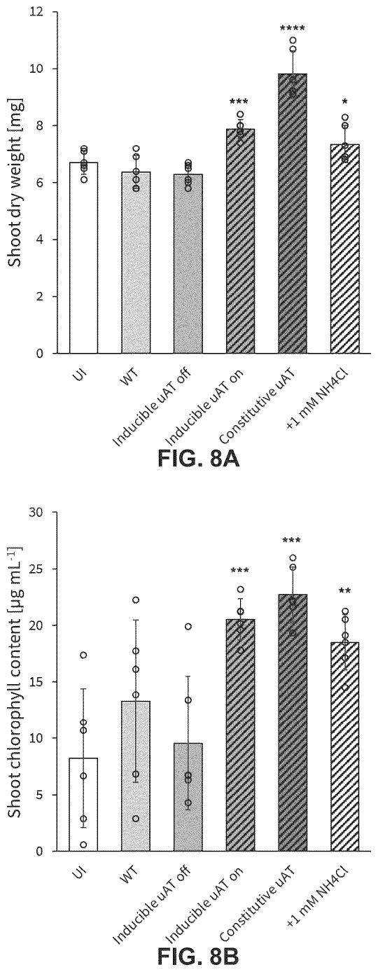

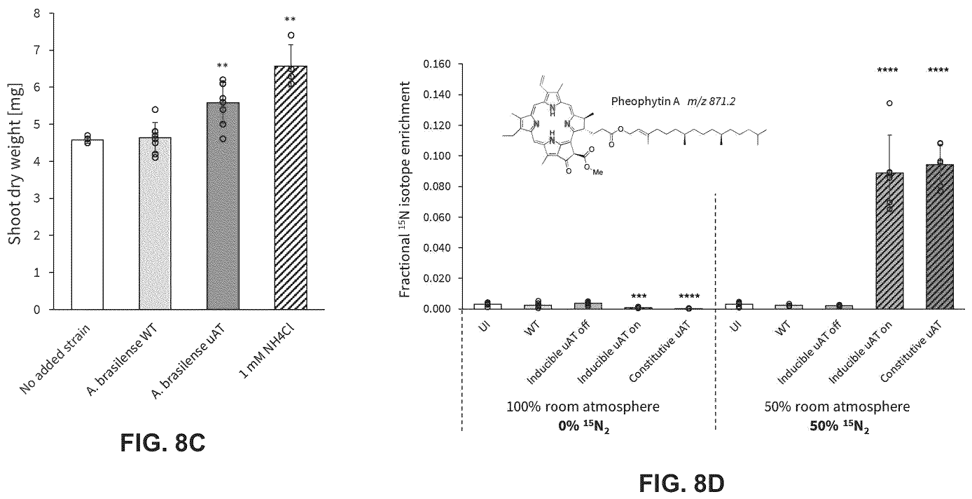

Turning to FIGS. 8A-8C, a number of embodiments are capable of increasing plant growth. In particular, FIGS. 8A and 8B illustrate shoot dry weight and shoot chlorophyll content, respectively, of plants grown in media with uninoculated diazotrophs (UI), wild type diazotrophs (WT), diazotrophs with an inducible uAT without induced expression (Inducible uAT off), diazotrophs with an inducible uAT with induced expression (Inducible uAT on), diazotrophs with a constitutive uAT (Constitutive uAT), and plants supplemented with NH₄Cl. Similarly, FIG. 8C illustrates plants grown in a nitrogen-poor soil mimic with a complex microbial community (e.g., background soil bacteria, fungi, etc.) inoculated with no additional microbial strain (No added strain), a wild type diazotroph (A. brasilense WT), a diazotroph with constitutive uAT (A. brasilense uAT), and supplemented with NH₄Cl. FIGS. 8A-8C illustrate that diazotrophs with uATs of many embodiments are capable of increasing plant growth and chlorophyll content, indicating better plant health and growth, even in environments where resource competition and ammonia scavenging may exist.

Turning to FIG. 8D, the increased plant growth is due to nitrogen fixation from embodiments of diazotrophs with uAT. Specifically, FIG. 8D illustrates the fractional ¹⁵N isotope enrichment in pheophytin, a plant-specific chlorophyll derivative. FIG. 8D shows plants grown in a standard atmosphere and plants grown in an atmosphere containing 50% ¹⁵N₂, where pheophytin of plants inoculated with embodiments of diazotrophs with constitutive uAT (Constitutive uAT) and inducible uAT with induced expression (Inducible uAT on) possess an amount of ¹⁵N, indicating that the ammonia produced in embodiments is bio-available to plants.

### Pre-Transcriptional GS Deactivation

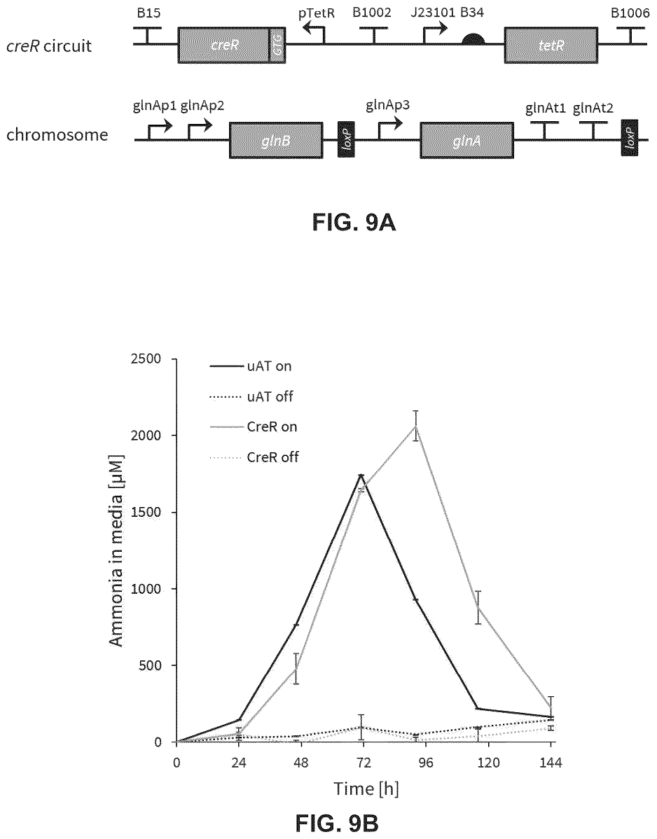

FIGS. 9A-9B illustrate systems for pre-transcriptional control of GS in accordance with some embodiments. In particular, FIG. 9A illustrates a system of pre-transcriptional control of a GS in accordance with a variety of embodiments. In certain pre-transcriptional control embodiments, Cre recombinase (CreR) is used to excise the GS encoding gene (e.g. g/nA) from a diazotroph. In some of these embodiments, an expression system containing the Cre recombinase is introduced into a diazotroph. Further, these embodiments will also introduce flanking IoxP sites (SEQ ID NO: 10), where a IoxP sequence (SEQ ID NO: 10) is introduced before and after the GS encoding gene. It should be noted that a system, such as illustrated in FIG. 9A could be combined with a multi-gene system, such as those illustrated in FIGS. 3E-3G to increase stability of certain diazotrophs. Certain embodiments will introduce IoxP sites flanking all GS genes, not just g/nA, within an organism to prevent the synthesis of glutamine and maintain accumulation of ammonia. Further embodiments will delete all but one GS within a diazotroph and flank the one remaining GS with IoxP sites. Additional embodiments will use additional recombinase systems known in the art to control GS expression.

FIG. 9B illustrates the ammonia concentration of inducible uAT and inducible CreR diazotrophs in accordance with certain embodiments. As illustrated, when the inducible uAT and inducible CreR embodiments are not induced, ammonia concentration remains close to zero, as GS is active, and ammonia is used to synthesize glutamine. However, when the inducible uAT or inducible CreR are activated using an inducer (e.g., tetracycline), ammonia concentration increases to a peak of approximately 1.7 mM at 72 hours in the inducible uAT embodiments, while the ammonia concentration peaks at approximately 2 mM at 96 hours in the inducible CreR embodiments.

### Methods of Increasing Bio-Available Nitrogen in Soil

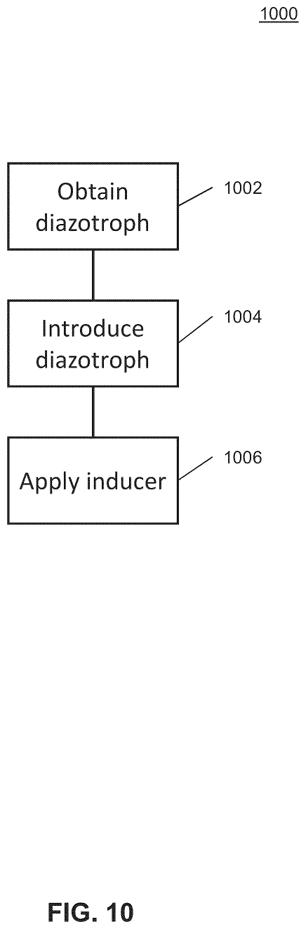

Turning to FIG. 10, a number of embodiments are directed to methods of increasing bio-available nitrogen in soil. In particular, FIG. 10 illustrates a method 900 to increase bio-available nitrogen. At step 1002, many embodiments will obtain a diazotroph in accordance with embodiments described herein. In certain embodiments, the diazotroph is capable of post-transcriptionally deactivating GS via adenylylation, while additional embodiments will deactivate GS pre-transcriptionally, such as by excising a GS gene. Certain embodiments will possess an inducible system for deactivating GS, while some embodiments will constitutively deactivate GS. Embodiments of post-transcriptional and excising diazotrophs are described elsewhere within this disclosure. A number of embodiments will obtain more than one diazotroph species.

At Step 1004, numerous embodiments will introduce a diazotroph in accordance with embodiments described herein to an agricultural media. Different types of media exist for use in embodiments, including media such as soil, potting mix, or growth media. In accordance with various embodiments, agricultural media includes media in an agricultural field, a greenhouse, a growth chamber, an agricultural hydroponic system, and/or or any other environment for growing plants. A number of embodiments will introduce the diazotrophs in any suitable method, such as by spraying liquid culture to the soil, seed coatings, applying pelletized or dry formulations of the diazotrophs to the soil. Certain embodiments will apply the diazotrophs before planting crops in the soil, while some embodiments will apply the diazotrophs after planting crops in the soil. Certain embodiments will apply diazotrophs multiple times during a plant growth season, such that multiple applications will occur periodically through a season. Certain embodiments will apply multiple types of diazotrophs (e.g., post-transcriptionally deactivating GS and excising diazotrophs) within a single application, while some embodiments will apply different types of diazotrophs at different times during a plant growth season. One of skill in the art will understand application methods to apply the diazotrophs to soil through various methods, including via air, tractor, manual, etc. spraying methods.

Many embodiments will apply an inducer to the soil at step 1006. In certain embodiments, the inducer activates a diazotroph with an inducible mechanism, such as an inducible uAT, such as those described herein. In various embodiments the inducer will be applied as a liquid, while some embodiments will apply the inducer dry. One of skill in the art will understand application methods to apply an inducer to soil through various methods, including via air, tractor, manual, etc. spraying methods. In other embodiments, diazotrophs will be induced prior to application, or induced by removal of a small molecule.

While FIG. 10 illustrates a method to increase plant growth and production, one of skill in the art would understand that certain steps may be performed in a different order, simultaneously, and/or omitted in accordance with the specifics of certain embodiments. For example, when using a constitutive embodiment, application of an inducer would not be necessary to activate any gene within a diazotroph. Additionally, certain embodiments will apply the inducer simultaneously with an inducible system, when immediate activation of a gene of interest is desired by a user.

### Exemplary Embodiments

Although the following embodiments provide details on certain embodiments of the inventions, it should be understood that these are only exemplary in nature, and are not intended to limit the scope of the invention.

### Example 1: Engineering Diazotrophs Produce Ammonia

BACKGROUND: Native diazotrophs will produce and consume ammonia to generate glutamine. Production of ammonia for exogenous use will require custom diazotrophs to prevent the consumption of ammonia by the diazotroph.

METHODS: All plasmids were assembled following standard protocol using Phusion polymerase and Gibson assembly. A PCR extension time of 24 s/kb was found to work best for high GC content assemblies. Standard synthetic parts from the BioBrick library were used in all genetic circuits. NEB E. coli DH5 alpha cells were used for plasmid propagation. Replicative plasmids (e.g., FIG. 4A) were transformed into A. brasilense Sp245 by electroporation of 1000 ng of DNA at 1.25 kV/cm and 200Ω and 25 μF. A brasilense electrocompetent cells were prepared as follows: cells were grown to OD 0.5 in LB media at 30° C. and 300 rpm, then rapidly cooled on ice and pelleted; cells were triple washed with ice cold 10% glycerol and flash frozen at high concentration in liquid nitrogen. Chromosomally integrative plasmids (e.g., FIG. 4B) were introduced by conjugation into A. brasilense by triparental mating using E. coli DH5 alpha as the donor strain and E. coli RK600 as the helper strain. For conjugation, all stains were grown on LB agar plates at 37° C. overnight. During grow-up, the donor plasmids were retained with 50 μg/mL kanamycin and the helper plasmid with 100 μg/mL chloramphenicol. Cells were scraped from plates at a ratio of 5:1:1 recipient to helper to donor, resuspended in 1 mL LB, pipette mixed, pelleted, dripped at high concentration onto antibiotic free LB agar, and incubated at 37° C. overnight. Transformants were selected on LB agar with 50 μg/mL kanamycin and 10 μg/mL streptomycin and re-streaked 3 times to ensure proper isolation. For double homologous recombination, negative selection was performed with 5 mM 4-chlorophenylalanine in minimal NFbHP media with 5 mM glutamine as the nitrogen source. NFbHP media contained 5 g/L sodium lactate, 1.7 mM sodium chloride, 811 μM magnesium sulfate, 180 μM calcium chloride, and 72 μM iron sulfate. Phosphates were stored separately and added to 34.4 mM dipotassium phosphate and 29.4 mM monopotassium phosphate. Trace elements were added to 1× from the following 50× stock solution: 4.5 mM boric acid, 1.4 mM manganese sulfate, 826 μM sodium molybdate, 86 μM zinc sulfate, and 32 μM copper sulfate. Colonies post-conjugation were inoculated until growth was observed (2-5 days) at 37° C. and 300 rpm, plated on antibiotic free LB, and screened by PCR for successful double recombination. All strains were stored at −80° C. in 1:1 LB to glycerol.

To assay GS activity, A. brasilense strains were plated on LB agar plates from glycerol stocks and grown at 30° C. for 2 days. Single colonies were then inoculated into 5 mL NFbHP media with 5 mM glutamine and grown for overnight at 30° C. and 300 rpm. Cultures were pelleted, triple washed with 1% potassium chloride, and inoculated at OD600 0.1 (unless noted otherwise) at a volume of 3 mL in semisolid NFbHP in 12 mL polypropylene culture tubes. NFbHP was turned semisolid by addition of 0.175% agar. Plasmids were retained with 50 μg/mL of kanamycin at all steps. Glutamine synthetase γ-glutamyl hydroxamate activity was determined based on previously reported procedures. (See e.g., Goldberg, R. B. & Hanau, R. Relation between the adenylylation state of glutamine synthetase and the expression of other genes involved in nitrogen metabolism. J Bacteriol 137, 1282-1289 (1979); the disclosure of which is incorporated by reference herein in its entirety.) All buffers were prepared fresh. After 24 h, cells were permeabilized by addition of 0.1 mg/mL CTAB and 0.25 mM manganese chloride at room temperature for 5 minutes, and then pelleted. Pellets were washed with ice-cold 1% potassium chloride. Per strain, 6 tubes of each 3 mL culture were combined and concentrated to 4 mL and stored at 4° C. until analysis. 5× concentrated assay mixture was prepared as follows: 675 mM imidazole hydrochloride buffer, 125 mM potassium arsenate, 100 mM hydroxylamine hydrochloride, 1.25 mM manganese chloride, 0.5 mg/mL CTAB. The pH was adjusted to 7.50 at 30° C., the isoactivity point of A. brasilense GS and GS-AMP in this assay as reported previously. (See e.g., Pirola, M. C., et al. Isolation and characterization of glutamine synthetase from the diazotroph Azospirillum brasilense. International Journal of Biochemistry 24, 1749-1754 (1992); the disclosure of which is incorporated by reference herein in its entirety.) 4× concentrated start mixture was made to 300 mM glutamine and the desired ADP concertation. Then, in 96 well format, 60 μL of 5× assay mixture was added to 150 μL of cell concentrate and equilibrated at 30° C. for 5 minutes. Reactions were initiated by addition of 75 μL of 4× start mix and took place under static conditions at 30° C. At each time point (0, 5, 10, and 15 minutes), 66 μL of the reactions was pipetted into a plate prepared with 133 μL stop mix containing 55 g/L iron chloride, 20 g/L trichloroacetic acid, and 21 mL/L concentrated hydrochloric acid. Absorbance at 540 nm was measured using the Synergy HTX plate reader. All reactions were prepared in n=4 technical replicates. Standard curves were determined by adding known concentrations of γ-glutamyl hydroxamate to the stop mix. Rates of γ-glutamyl hydroxamate formation were normalized to total protein content in reactions that was determined using the standard Bradford assay on the cell concentrate.

To assay ammonia production, A. brasilense strains were cultured as described for the GS activity assay. Inducible strains were switched on within 30 minutes post inoculation by addition of 200 ng/mL anhydrotetracycline, unless noted otherwise. Cultures were incubated statically at 30° C. Samples of 200 μL were taken at time points and pelleted. 120 μL of supernatant was stored at −20° C. until analysis. 20 μL of each sample was analyzed in 96-well PCR plate format using a modified version of the previously described indophenol procedure: 120 μL of 150 mM sodium acetate pH 3.7 was added, followed by 80 μL of saturated chlorine water and 30 μL of freshly prepared 8% phenol in water. (See e.g., Bolleter, W. T., et al. Spectrophotometric Determination of Ammonia as Indophenol. Analytical Chemistry 33, 592-594 (1961); the disclosure of which is incorporated herein in its entirety.) Plates were immediately incubated at 95° C. for 5 minutes in a thermocycler and then rapidly cooled in cold water. Color was developed by addition of 100 μL of 500 mM borate buffer pH 12.5. 200 μL of each reaction was transferred to a clear bottom 96-well polystyrene plate and analyzed for absorbance at 625 nm using the Synergy HTX plate reader. On-plate standards were run for every plate using uninoculated reaction buffer and ammonium chloride as the standard. Mutants were monitored by plating ammonia producing cultures onto LB again and analyzing individual colonies by PCR at loci of interest.

For NMR analysis, strains were prepared as for the ammonia production assay with the following modifications: culture volumes were 5 mL in 25 mL scintillation vials capped with a rubber stopper. The headspace was replaced with 20 mL ¹⁵N₂ gas, ¹⁴N₂ gas, or Argon. Then 5 mL was displaced with 02 and vials were incubated at 30° C. for 65 h. Following incubation, cultures were uncapped, pelleted, and the supernatant frozen at −20° C. until analysis. Samples were quantified for ammonia using the indophenol method as described, and prepared for ¹H-NMR analysis by addition of 50 μL deuterated DMSO and 25 μL concentrated HCl to 425 μL sample. Standards were prepared equivalently, using 10 mM ¹⁴NH₄C1 and 10 mM ¹⁵NH₄C1 in uninoculated culture buffer. Spectra were collected using an INOVA 600 Oxford NMR with a 64-scan selective pulse sequence for ammonia as reported previously. (See e.g., Nielander, A. C. et al. A Versatile Method for Ammonia Detection in a Range of Relevant Electrolytes via Direct Nuclear Magnetic Resonance Techniques. ACS Catalysis 9, 5797-5802 (2019); the disclosure of which is incorporated herein in its entirety.)

RESULTS: As illustrated in FIG. 5A, ammonia was not detected in the media of the WT strain—as expected—but several of the uAT expressing A. brasilense Ag/nE strains did result in 0.3-3 mM levels of ammonia accumulation over 2-4 days. As previously described, GS is a homododecamer and has 12 total adenylylation sites, thus the maximal adenylylation (shutdown) state is 12. FIG. 5B illustrates that ammonia accumulates in the media when average adenylylation levels exceed 11—see strains, uAT4, AT-C, and ATC3. uAT2 and uAT5, conferred slightly lower adenylylation levels of 9 and did not lead to detectable levels of ammonia. These data confirm that the inability to assimilate ammonia through GS deactivation results in its extracellular release, and suggest that an average adenylylation threshold between 9 and 11 is required to sufficiently deactivate GS for ammonia accumulation. Additionally, FIG. 7C illustrates improved evolutionary stability in strains possessing multiple copies of an inducible uAT.

FIG. 3C illustrates an A. brasilense Ag/nE embodiment carrying an inducible circuit with an AT-C(SEQ ID NO: 1) uAT in nitrogen free media under controlled atmosphere conditions of 20% 02 and 80% of either ¹⁴N₂, ¹⁵N₂, or Argon. Since commercial ¹⁵N₂ gas often contains small amounts of ¹⁵NH₃, the quantitative indophenol assay was performed to verify that no appreciable amounts of ammonia were in the media above the assay detection threshold of 10 μM (FIG. 6A). Next, media of inoculated vials was analyzed using proton nuclear magnetic resonance (¹H-NMR) with a previously reported pulse sequence optimized for the detection of ammonia. (See e.g., Nielander, A. C. et al. ACS Catalysis 9, 5797-5802 (2019) (cited above).)¹H-NMR is ideally suited for this kind of analysis as isotopically unique differences in nuclear spin manifest as distinctive coupling constants inherently observed by NMR techniques. Thus, ¹⁴NH₄⁺ and ¹⁵NH₄⁺ are readily distinguishable owing to the different nuclear spins of each isotope. ¹⁴NH₄⁺, with a spin=1, appears as a 1:1:1 triplet with ¹J_{H-N}=51 Hz while ¹⁵NH₄⁺, with a spin=1/2, appears as a 1:1 doublet with ¹J_{H-N}=72 Hz. We find that the ¹⁵N₂ atmosphere samples do indeed show a predominant doublet with ¹J_{H-N}=73 Hz, from which we conclude that ammonia in the media is de novo fixed from gaseous nitrogen (FIG. 6B).

AT-C(SEQ ID NO: 1) on the inducible circuit illustrated in FIG. 3C from plasmid pTS7: in the same nitrogen fixing conditions as before, no ammonia production was detected (off-state) until the inducer concentration reaches 20-40 ng/mL anhydrotetracycline, above which ammonia production is constant (on-state) with respect to inducer concentration at average rates of 50 μM h⁻¹ for approximately 70 hours (FIGS. 7A-7B). The inducible on-state ammonia production rate is twice as high and lasts twice as long as the equivalent constitutive strain performance FIG. 7B). Ammonia production rates were found to scale robustly with OD but require sufficient surface area for gas exchange with the atmosphere at higher OD.

CONCLUSION: This proof-of-concept study demonstrates the ability to engineer diazotrophs to produce ammonia, which can be released into solution.

### Example 2: Plant Growth Enhancement

BACKGROUND: Plants, especially many crops, will benefit from additional sources of bio-available nitrogen, such as ammonia. Ammonia producing diazotrophs, such as these embodiments, may provide a beneficial alternative to industrially generated nitrogen fertilizers.

METHODS: S. viridis A10 seeds were first surface sterilized in 10% bleach and 0.01% Tween-20 for 10 minutes, followed by a triple water wash, and then imbibed overnight at 30° C. ⅕ strength NFbHP with addition of 1 mM NH₄Cl, 0.5 mM calcium chloride, and 2% w/v sucrose was prepared to a semisolid state (0.175% agar) as described. Post imbibition, seeds were planted on 1.2 cm² PTFE mesh floating on 10 mL media in 50 mL capped glass tubes. Growth chamber conditions were as follows: 16 h light cycles with 30° C. light and 24° C. dark temperatures, at a relative humidity of 50%. Strains were prepared as described for the ammonia assay and inoculated 14 days post planting to an OD600 of 0.1. Anhydrotetracycline was added to the relevant experimental groups at 200 ng/mL following inoculation. 14 days post inoculation, whole shoots were harvested and lyophilized overnight prior to dry weight determination.

At 14 days post planting—coincident with microbial inoculation—50% of the headspace in the label group was displaced with ¹⁵N₂ gas. Post lyophilization and dry weight collection, chlorophyll of shoots was extracted similar to previous reported methods. (See e.g., Kahn, M. et al. A mass spectrometry method for measuring N-15 incorporation into pheophytin, Vol. 307. (2002); and Parra-Colmenares, A. & Kahn, M. L. Determination of nitrogen fixation effectiveness in selected Medicago truncatula isolates by measuring nitrogen isotope incorporation into pheophytin. Plant and Soil 270, 159-168 (2005); the disclosures of which are incorporated herein in their entireties.) Whole shoots were treated as individual samples. Each sample was homogenized at 25 Hz for 2 minutes and extracted in 1.4 mL methanol for 2 h at room temperature. Leaf fragments were precipitated by centrifugation and 1 mL of supernatant was transferred to a new tube. 145 μL dioxane and 180 μL water were added and samples were chilled at −80° C. for 2 h. Chlorophyll was pelleted at 21,000 rcf for 15 minutes at 4° C., resuspended in 200 μL 1:1 methanol to acetone, and stored in this state for several days at −80° C. until analysis. Chlorophyll was then converted to pheophytin by addition of 1 μL concentrated hydrochloric acid and clarified for 2 minutes at 21,000 rcf. Standards were prepared from a chlorophyll authentic standard following the same pheophytin conversion method. Pheophytin isotope abundances were analyzed by quantitative time-of-flight (qTOF) mass spectrometry on an Agilent 6545 LC-MS. For this, 5 μL of sample was directly injected into the MS in electrospray ion positive mode with a fragmentor voltage of 175 V and capillary voltage of 3500 V. 10% water in acetonitrile and 0.1% formic acid was used as the solvent at a flowrate of 0.6 mL/min with a sheath gas flow of 12 L/min at 300° C., drying gas flow of 12 L/min at 250° C., and a nebulizer pressure of 10 psi. Mass-isotopomer distributions (MIDs) were computationally extracted from raw data as previously reported. (See e.g., Nett, R. S. et al. D2O Labeling to measure active biosynthesis of natural products in medicinal plants. AIChE Journal 64, 4319-4330 (2018); the disclosure of which is incorporated herein in its entirety.)

For complex community analyses, S. viridis A10 seeds were surface sterilized as described and planted on 5 mL calcined clay under the same growth conditions as described for minimal media methods. Soil filtrate was prepared by adding 10 mL of Horticulture 713405 HP Pro-Mix soil to 100 mL of water and stirred for 30 minutes at room temperature. 2 mL of this broth was applied to each tube at the time of planting. A. brasilense strains were cultured and prepared as described for minimal media methods and applied 9 days post planting as 500 μL of OD600 of 1 to each tube. Plants were harvested and processed as described in minimal media methods at 28 days post planting.

RESULTS: FIG. 8A illustrates significant growth promotion with both constitutively producing and inducible on-state strains at 54% and 24% over WT inoculated plants respectively. The wild type and inducible off-state inoculated samples showed no growth promotion, suggesting no native nitrogen transfer. In FIG. 8B, total chlorophyll content was also determined as metric for plant health: the results mirror dry weight conclusions, with an increase of 71% and 55% over WT for the constitutive and inducible on-state strains respectively. (See e.g., Liang, Y. et al. A nondestructive method to estimate the chlorophyll content of Arabidopsis seedlings. Plant Methods 13, 26 (2017); the disclosure of which is incorporated herein in its entirety.)

In complex microbial communities, no statistically significant difference between adding no additional strains at 9 days or adding A. brasilense WT was observed. Introduction of the constitutive uAT expressing chromosomal integration strain lead to a growth promotion of 20% over WT (p=0.001) (FIG. 8C). Even though a complex community background might limit strain performance through resource competition and ammonia scavenging, statistically significant growth promotion was still observed, which demonstrate practical promise for engineered biological nitrogen fixation.

FIG. 8D illustrates the incorporation of ¹⁵N into pheophytin. In the 50% ¹⁵N₂ displaced atmosphere, no isotope enrichment above the ¹⁵N natural abundance (0.4%) for uninoculated, WT, or inducible-off inoculated plants was observed. For the constitutive and inducible-on inoculated plants, 9.4±1.3% and 8.9±2.5% integration of ¹⁵N atoms in the total N of pheophytin at high statistical significance (p<10⁻⁴) was observed. This method is also precise enough to detect a statistically significant dilution of natural ¹⁵N isotopes in plants grown under regular atmospheric conditions and inoculated with ammonia producing strains (p<10⁻³). This isotope dilution occurs under significant de novo gaseous nitrogen influx because natural abundances of ¹⁵N in biomass are higher than in the atmosphere. Taken together, the data demonstrate direct transfer of nitrogen gas to plants cultivated in the presence of engineered A. brasilense.

CONCLUSION: Various embodiments are capable of increasing plant growth and health by inoculation of media and soil with engineered diazotrophs capable of fixing nitrogen. Various embodiments show direct transfer for nitrogen gas from the air to plant molecules.

### Doctrine Of Equivalents

Having described several embodiments, it will be recognized by those skilled in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Those skilled in the art will appreciate that the presently disclosed embodiments teach by way of example and not by limitation. Therefore, the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

TABLE 1
uATs in accordance with embodiments

| Name | Species | Amino Acid Range | SEQ ID NO |
|---|---|---|---|
| AT-C | E. coli | 423-946 | 1 |
| ATC3 | E. coli | 609-946 | 2 |
| uAT1 | A. brasilense | 449-1003 | 3 |
| uAT2 | A. brasilense | 625-1003 | 4 |
| uAT3 | A. brasilense | 481-1003 | 5 |
| uAT4 | A. brasilense | 506-1003 | 6 |
| uAT5 | A. brasilense | 529-1003 | 7 |

What is claimed is:

1. An inducible genetic circuit to deactivate glutamine synthetase in an organism comprising:
a unidirectional adenylyl transferase capable of adenylylating glutamine synthetase; and
a system for inducing expression of the unidirectional adenylyl transferase enzyme.

2. The inducible genetic circuit of claim 1, wherein the unidirectional adenylyl transferase is selected from the SEQ ID NOs: 1-7.

3. The inducible genetic circuit of claim 1, wherein the unidirectional adenylyl transferase is SEQ ID NO: 3-7.

4. The inducible genetic circuit of claim 1, wherein the system for inducing expression is a tetracycline inducing system.

5. The inducible genetic circuit of claim 1, further comprising a second unidirectional adenylyl transferase capable of adenylylating glutamine synthetase.

6. A method of increasing bio-available nitrogen in agricultural environment comprising:
obtaining an engineered diazotrophic organism, wherein the engineered diazotrophic organism contains a genetic circuit capable of deactivating glutamine synthetase within the diazatrophic organism; and
introducing the engineered diazotrophic organism into an agricultural media.

7. The method of claim 6, wherein the diazotrophic organism comprises a genetic circuit to allow expression of an enzyme capable of deactivating glutamine synthetase within the diazatrophic organism, wherein the genetic circuit is integrated into chromosomal DNA of the diazatrophic organism.

8. The method of claim 7, wherein the genetic circuit comprises:
a unidirectional adenylyl transferase capable of adenylylating glutamine synthetase; and
a system for inducing expression of the unidirectional adenylyl transferase enzyme.

9. The method of claim 7, wherein the unidirectional adenylyl transferase is selected from the SEQ ID NOs: 1-7.

10. The method of claim 7, wherein the unidirectional adenylyl transferase is SEQ ID NO: 3-7.

11. The method of claim 7, wherein the system for inducing expression is a tetracycline inducing system.

12. The method of claim 7, wherein the genetic circuit further comprises a second unidirectional adenylyl transferase capable of adenylylating glutamine synthetase.

13. The method of claim 6, further comprising applying an inducer to the engineered diazotrophic organism.

14. The method of claim 13, wherein the inducer is tetracycline.
